(19) United States
(12) Patent Application Publication
(10) Pub. No.: US 2006/0287614 A1
(43) Pub. Date: Dec. 21, 2006

(54) Testing therapy efficacy with extremity and/or joint attachments

(75) Inventors: Neville Hogan, Sudbury, MA (US); Hermano Igo Krebs, Cambridge, MA (US); Bruce T. Volpe, Bronx, NY (US)

Correspondence Address: FOLEY HOAG, LLP;PATENT GROUP, WORLD TRADE CENTER WEST, 155 SEAPORT BLVD, BOSTON, MA, 02110, US

(73) Assignees: Cornell Research Foundation, Inc., Ithaca, NY (US); Massachusetts Institute of Technology, Cambridge, MA (US)

(21) Appl. No.: 11/154,197

(22) Filed: Jun. 16, 2005

(51) Int. Cl. A61B 5/117

(52) U.S. Cl. 600/595; 600/587

(57) ABSTRACT

A method of measuring a change in neurological and/or muscular performance of a subject may include attaching an attachment to a subject. The subject may then be directed to perform a motion. A first force imposed by the subject on the attachment and/or a first motion of the attachment is sensed. The subject's first response may then be compared to a second response to determine a change in the subject's neurological and/or muscular performance.

### SUMMARY

The disclosed subject matter relates to testing the efficacy of a therapeutic treatment by using robotic-type devices that act on or measure motion of various extremities and joints of a subject, such as an animal or a human, to measure changes in neurological and/or musculoskeletal performance.

In an embodiment, a system for measuring a change in neurological and/or muscular performance of a subject may include a joint and/or extremity attachment. The attachment may include a support so sized and shaped as to be able to receive a portion of the subject's anatomy, a second support so sized and shaped as to be able to receive another portion of the subject's anatomy, and a linkage connecting the two supports. The system may also include a sensor producing a first signal indicative of a force imposed by the subject on the attachment and/or of a motion of the attachment. The system may further include a controller which may include a computation circuit, responsive to the first signal, to compare the first signal to a second signal. The second signal may be produced at a different time from the first signal, the second signal being indicative of a force imposed by the subject on the attachment and/or of a motion of the attachment. The computation circuit may generate an output signal indicative of a difference, if any, between the first signal and the second signal.

In an embodiment, the attachment may be an upper-extremity attachment including a wrist attachment. The wrist attachment may include a forearm support, so sized and shaped as to be able to receive a forearm of the subject, the forearm support defining a long axis. A handle may be included that is so positioned in relation to the forearm support and so sized and shaped as to be able to receive the subject's hand. A transmission system may also be included to provide rotation with at least three degrees of freedom.

In another embodiment, the upper-extremity attachment may further include a shoulder-elbow motion device. This may contain a member assembly having at least one degree of freedom and a distal free end to which the wrist attachment is coupled with at least one degree of freedom.

In another embodiment, the attachment may be an ankle interface. The ankle interface may include a leg connection attachable to a user's leg and a foot connection attachable to the user's corresponding foot. The ankle interface may also include a transmission system coupling the leg connection and the foot connection with at least two degrees of freedom.

In another embodiment, a method of conducting an efficacy study may include selecting first and second groups of subjects and measuring a pre-administration property of each subject by attaching an attachment to each subject and sensing a property indicative of each subject's neurological and/or musculoskeletal function. A first therapeutic treatment may be administered to the subjects in the first group, and a second therapeutic treatment, a placebo treatment, or no treatment may be administered to the subjects of the second group. After administration, a post-administration property of each subject may be measured by attaching an attachment to each subject and sensing a property indicative of each subject's neurological and/or musculoskeletal function. The pre- and post-administration properties measured from subjects in the first group may be compared to determine an efficacy of the first therapeutic treatment. The pre- and post-administration properties measured from subjects in the second group may be compared to determine an efficacy of the second therapeutic treatment, a placebo treatment, or no treatment. The efficacy of the first therapeutic treatment may be compared to the efficacy of the second therapeutic treatment, a placebo treatment, or no treatment.

### BRIEF DESCRIPTION OF THE DRAWINGS

FIG. 1 depicts an exemplary embodiment of a wrist attachment device.

FIG. 2 depicts an exemplary embodiment of a shoulder-elbow-wrist attachment device.

FIG. 3 depicts an exemplary embodiment of an ankle interface attachment device.

FIGS. 4-5 depict schematic representations of systems for determining a change or difference in neurological or muscular performance.

FIG. 6 depicts a flow chart of an exemplary method for determining a change or difference in neurological or muscular performance.

FIG. 7 depicts a flow chart of an exemplary method of conducting an efficacy study.

### DETAILED DESCRIPTION

1. Overview

The systems and methods described herein can be used to determine a change in neurological or muscular performance of a subject, such as in response to drug therapy. The system includes a device that may be attached to a subject for the purpose of measuring the subject's ability to move. The device can also exert forces on the subject to provide assistance, perturbation and/or resistance to the subject's limb. The device is typically one that can be attached to a subject in such a way as to move or be moved by a portion of the subject's anatomy, such as an extremity or a joint. Examples of extremities and joints include but are not limited to neck, spine, pelvis, shoulder, arm, elbow, forearm, wrist, hand, metacarpal-phalangeal joints, interphalangeal joints, thigh, knee, leg, ankle, foot, metatarsal-phalangeal joints, and pedal interphalangeal joints, and combinations of these.

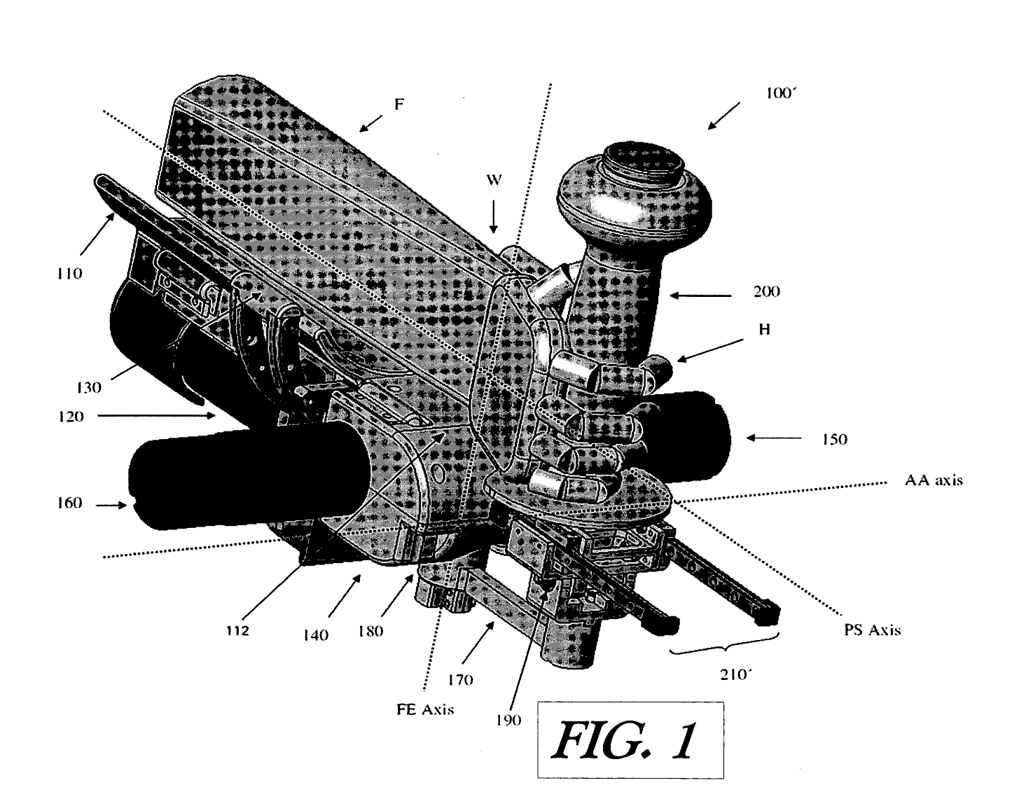

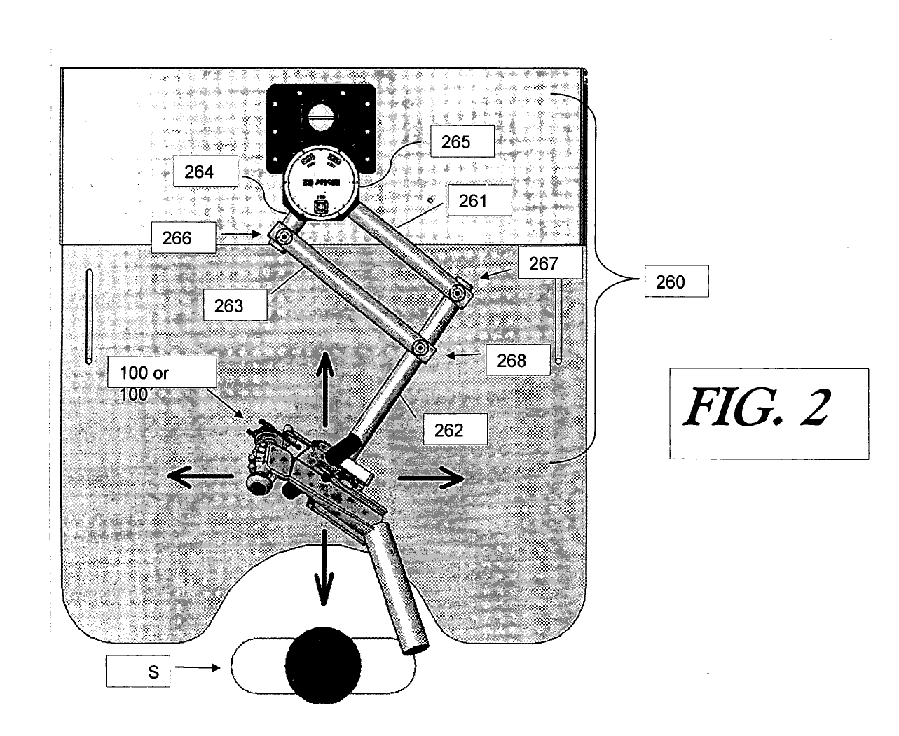

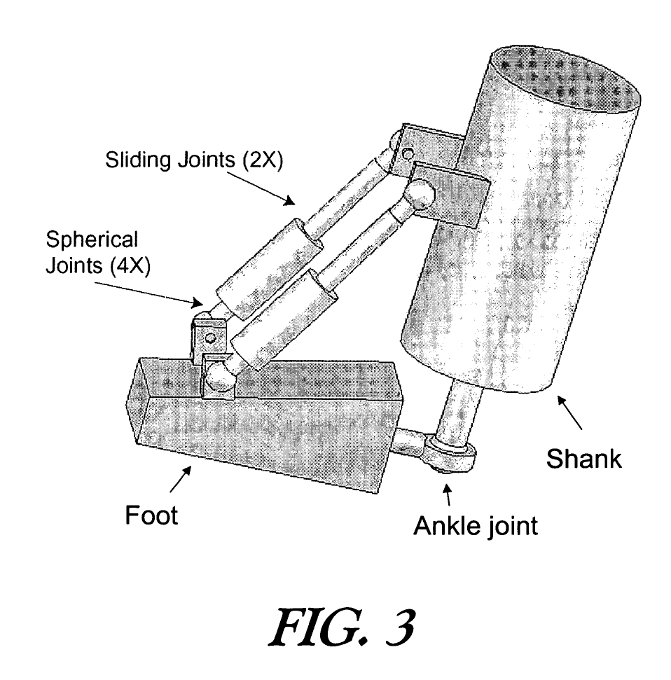

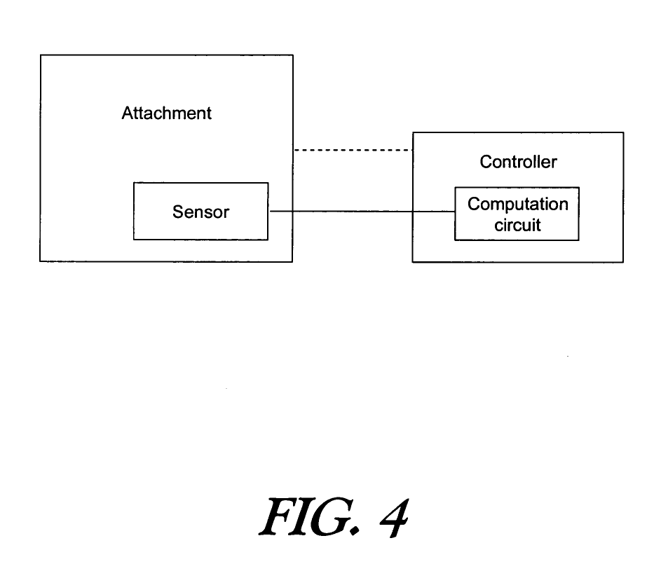

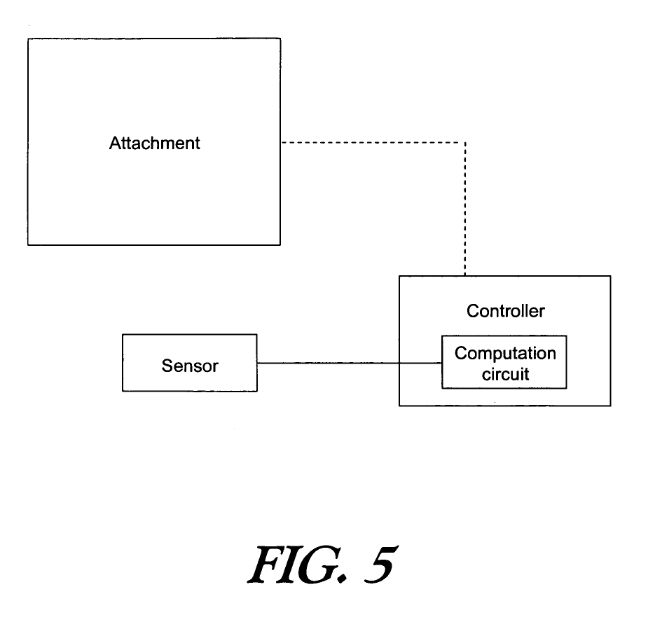

In one mode, the system may include an upper extremity attachment device such as the wrist attachment device of FIG. 1. The system may include a shoulder-elbow attachment device as shown in FIG. 2 or a shoulder-elbow motion device (element 260 in FIG. 2). Alternatively, the system may include an ankle interface attachment device such as that shown in FIG. 3. The ankle, wrist and/or shoulder-elbow attachment or motion devices may be used to measure neurological and/or musculoskeletal function of a subject. The systems may also include sensors on the attachment device to sense the response of a subject, and a controller which may include a computation circuit to determine the performance of a subject's neurological or muscular performance, as shown in FIG. 4. Alternatively, the system may include sensors that are not on the attachment device, as shown in FIG. 5.

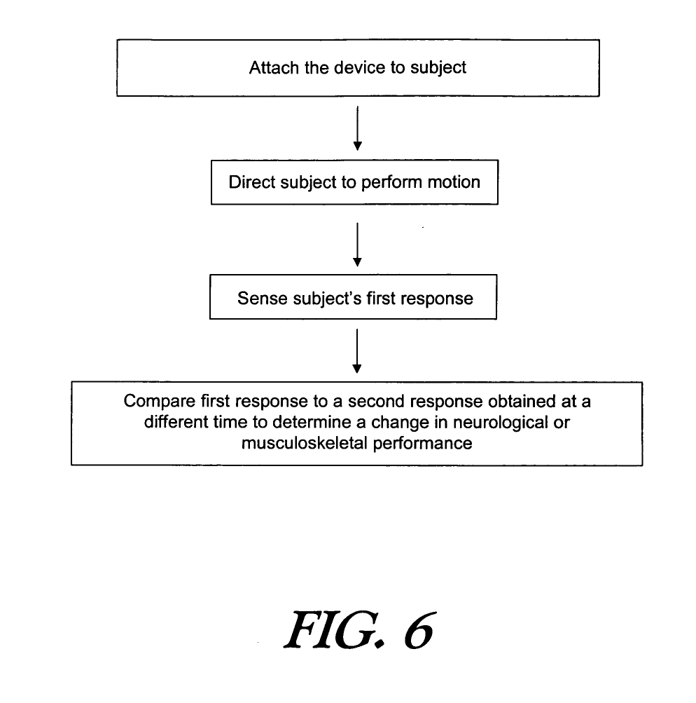

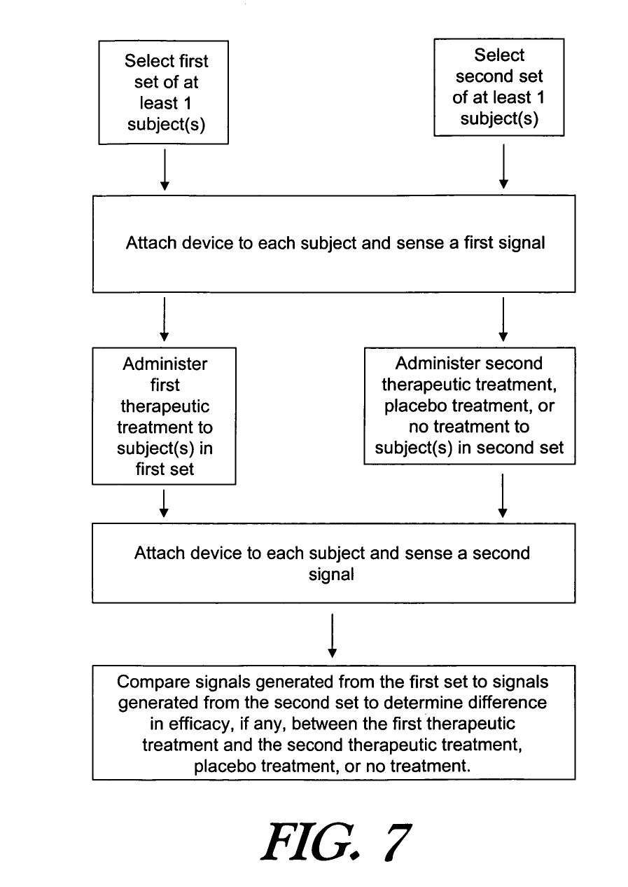

The system may use specific methods to determine a subject's neurological and/or muscular performance as shown in the flow chart of FIG. 6. The steps of the method may include attaching the device to the subject, directing the subject to perform a motion, sensing the first response of the subject, and comparing the first response to a second response to determine a change in the performance of the subject. More specifically, the system may be used to determine the efficacy of a therapeutic treatment. FIG. 7 depicts a flow chart to perform an efficacy study, including testing the efficacy of a therapeutic treatment in helping a subject recover from neurological and/or muscular pathologies. These uses are described in greater detail below.

2. Wrist and Shoulder-Elbow-Wrist Attachments

Wrist and shoulder-elbow-wrist attachments are extensively described in u.s. application Ser. No. 10/976,083 filed Oct. 27, 2004 by Krebs et al., the contents of which are hereby incorporated herein by reference. FIG. 1 depicts an exemplary embodiment of a wrist attachment 100. A subject's forearm (F), wrist (W), and hand (H) are shown in position on the attachment. The forearm F rests on the forearm support 110, the wrist W rests on the wrist support 112, and the hand H rests on the handle 200. The handle may also include a palm stop (not shown) to help prevent rotation of the hand around the handle. The forearm support may be so sized and shaped as to be able to receive the forearm of a subject from above without obstruction. For example, the depicted embodiment has no structure above the forearm support, so that a patient's forearm can be lowered directly onto the forearm support without having to navigate the forearm around structural elements. This non-obstructed configuration facilitates forearm placement and can reduce the mounting time compared to other systems.

A wrist attachment may also include several motors and linkages in order to apply various torques to a wrist that is positioned in the wrist attachment. The attachment may include a pronation/supination (PS) motor 120. The PS motor can be mounted to the forearm support 110 in the depicted embodiment, but this is not necessary. The PS motor may be coupled to a PS ring 130 so that the PS ring rotates in a PS plane (not shown) and about a PS axis (shown in FIG. 1) in response to actuation by the PS motor. As the names suggest, the PS motor and PS ring impart torques to pronate and supinate an attached wrist. The wrist attachment may also include a differential mechanism 140 for imparting flexion/extension and abduction/adduction torques. The differential mechanism may be housed inside wrist support 112. The differential mechanism may be mounted or otherwise coupled to the PS ring, so that the differential mechanism is carried by the PS motor. Alternatively, the actuators can be disposed on serial linkages. A wide variety of actuators may be used. In some embodiments, brushless servomotors may be preferred due to their potential for higher torques, lower speeds, and better heat dissipation. The wrist position and motion can be measured by keeping track of the rotation states of the actuators. Rotational state feedback for the controller can be provided by incremental optical encoders mounted on each motor shaft. Encoders should be mounted without overconstraint in order to preserve the encoder signal.

The differential mechanism acts on an arm 170 that is coupled to the differential mechanism at joint 180. As discussed in greater detail below, the differential mechanism can cause the arm to rotate with two degrees of freedom: tilting up and down and swinging from side to side. These two degrees of freedom allow the wrist attachment to transmit abduction/adduction torques and flexion/extension torques, respectively, to an attached wrist. The arm may be coupled by pivot 190 and slider 210 to the handle 200. The pivot 190 on which the slider 210 is mounted allows a single degree-of-freedom of rotation about an axis perpendicular to both the long axis of the arm and the long axis of the handle. The slider may include one, two, or more arms to increase stability. A wrist attachment may also include various straps, buckles, or other restraining devices to help keep a subject's forearm, wrist, and/or hand safely secured.

FIG. 1 also shows three principal axes of motion for a wrist attachment. The pronation/supination (PS) axis extends parallel to the long axis of the device and is the axis about which the PS slide ring may rotate. Rotation of the device about the PS axis will cause or result from pronation and supination of the subject's wrist. The flexion/extension (FE) axis extends through the subject's wrist, the differential mechanism 140, and joint 180 perpendicular to arm 170. Rotation of the device about the FE axis will cause or result from flexion and extension of the subject's wrist. The abduction/adduction (AA) axis extends perpendicular to the FE axis and perpendicular to the arm 170 and passes through the differential mechanism 140. Rotation of the device about the AA axis will cause or result from abduction and adduction of the subject's wrist. The wrist attachment arm rotates about the AA axis, while the subject's wrist rotates about a different axis parallel to the AA axis, because the wrist W, of course, cannot be located in the same place as the arm 170. Slider 210 and pivot 190 allow for the misalignment of these axes.

FIG. 2 shows one embodiment of a shoulder-elbow-wrist attachment. A wrist of subject S may be positioned on a wrist attachment 100 as described above. The wrist attachment itself is coupled to a shoulder/elbow motion device 260. Shoulder/elbow motion devices, as well as several other motion devices, are described extensively in U.S. Pat. No. 5,466,213 to Hogan et al., the contents of which are hereby incorporated herein by reference. The shoulder/elbow motion device may include arm member 261, forearm member 262, third member 263, and fourth member 264. The arm member may be coupled at its distal end to the proximal end of the forearm member by an elbow joint 267. The arm member and the forearm member may be rotatable with respect to one another about the elbow joint. The third member may be coupled at its distal end to a position along the midshaft of the forearm member by an elbow actuation joint 268. The third member and the forearm member may be rotatable with respect to one another about the elbow actuation joint. The fourth member may be coupled at its proximal end to the proximal end of the arm member by a shoulder joint 265. The fourth member and the arm member may be rotatable with respect to one another about the shoulder joint. The fourth member may also be coupled at its distal end to the proximal end of the third member by a fourth joint 266, and the third member and the fourth member may be rotatable with respect to one another about the fourth joint. The four members may be oriented in a plane and be moveable in that plane. In some embodiments, the four members are rotatable in only that plane.

The shoulder/elbow motion device may also include a shoulder motor coupled to one of the joints and controlling motion of the shoulder joint. The shoulder/elbow motion device may further include an elbow motor coupled to one of the joints and controlling motion of the elbow actuation joint. The motors are not shown in FIG. 2, but in the depicted embodiment, both motors are located at shoulder joint 265. Locating the motors far from the end point can reduce inertia of the device. In some embodiments, the motors may be aligned along a vertical axis so that the effects of their weight and that of the mechanism is eliminated.

3. Ankle Interface

FIG. 3 depicts a kinematic mechanism for an ankle interface. The device includes a leg connection that attaches the interface to a user's leg. The leg connection may include one or more straps that extend around the user's leg to hold the device against the leg. The leg connection may include a knee-brace to help immobilize the device with respect to the knee and prevent motion of the device relative to the leg. The interface may also include a foot connection that receives the foot. The leg connection and the foot connection may be coupled to one another through a motor and transmission system. The motor and transmission system can develop forces to move the foot relative to the leg in various motions, such as dorsiflexion/plantar flexion and inversion/eversion. The device represented by the kinematic mechanism shown in FIG. 3 includes two sliding joints or actuators mounted in parallel with spherical joints on either end. This mechanism will allow actuation in dorsiflexion/plantar flexion and inversion/eversion.

Ankle interface devices are extensively described in U.S. Provisional Application No. 60/613,421 filed Sep. 27, 2004 by Krebs et al., the contents of which are hereby incorporated herein by reference.

4. Systems for Measuring Neurological and Muscular Performance

There is an urgent need for a complete cure for stroke, since over 90% of those who survive stroke will need to recover from some sensory motor impairment because of paralyzed joint and/or extremity function. The current population of 4.7 million stroke survivors will swell considerably over the next ten years from the combination of improved medical and emergency care leading to increased survival, and the aging “baby boom” generation. With a new stroke victim every 45 seconds, the potential for a group of neurologically injured patients in need of new treatment is huge. Standard of care for treating this aspect of stroke recovery is labor intensive and too costly in an era of shrinking health care budgets for chronic disabilities. Furthermore, while fundamental experiments should have provided a range of candidate new molecules to enhance neuro-plasticity, the market is woefully bare.

The standard approach for new drug development is to use animal models, and while end-points of stroke size or other biological surrogate markers may be useful, behavioral recovery dependent on sensory-motor improvement is too often peculiar, ad hoc, and without the validity and theoretical depth of understanding that is available in the recovery of memory performance based on hippocampal injury. A human experimental paradigm, where sensory motor measures can be accurately measured and fit into a construct of motor recovery and motor learning, so that fundamental experimental changes might be taken as clues to develop drug treatment programs alone or in concert with physical training protocols, can greatly enhance reliability and reproducibility of experimental data by eliminating the essentially unavoidable variability and subjectivity of assessments based on human observation. Animal paradigms can also be used to improve reliability and reproducibility of pre-clinical data. Such improvements may result, for example, in fewer numbers of experimental animals needed to establish statistically significant efficacy data.

Indicia of motor learning include, for example, saltation (abrupt performance improvements; this can be assessed observing the magnitude of change between measurements), ability to generalize movements beyond training or testing conditions (can be tested by comparing performance in disparate settings or tasks), order effect (can be tested by presenting test in specific, not random order), interference (can be assessed by testing subject in disparate ways close in time), and progressive blending (can be assessed by measuring change in the number of submovements performed by a subject in completing a complex movement).

The development of treatments with interactive robotic devices will lighten the disability burden for each patient, and, importantly, generate health-cost savings. Moreover, therapists who use the support of robotic treatment will increase their efficiency and improve the functional outcome of their patients. Attachment devices such as those described herein can be used to establish standardized and repeatable experimental paradigms to measure the effect of drug treatment on sensory and/or motor function, such as recovery from stroke.

FIGS. 4 and 5 provide schematic representations of exemplary systems. At the simplest, a system includes an attachment device and a controller. In use, the attachment device is attached to a subject. A sensor, either coupled to or otherwise part of the attachment device (FIG. 4) or separate from it (FIG. 5) produces a signal indicative of the subject's ability to move; this signal is sent to a computation circuit in a controller. In some embodiments, the controller may be coupled to the attachment (represented by the dotted line) to actuate the attachment, i.e., to cause the attachment to exert a force on the subject. Such actuation can be used to assist the subject in a motion or to challenge the subject's motion, such as by providing resistance to the subject's volitional movement or by perturbing the movement. As shown in FIG. 5, the sensor need not be part of the attachment. Instead, it may be a separate device or integrated with the controller. For example, the sensor could include one or more cameras or light detectors that track the position and orientation of the attachment device or a portion thereof. Other measurement systems include, for example, EEG and EMG. A system can be used to generate more than one signal, so that signals may be compared to one another. Such comparisons can be used, for example, to measure a change in one subject's neurological or musculoskeletal performance over time or in response to a therapy (such as drug therapy, physical therapy, or both), or to compare one subject's performance to another subject's.

Particular parts of a subject's body can be used to assess changes in neurological or musculoskeletal performance. This may involve various part of the subject's body, such as an upper extremity and/or a lower extremity. An attachment described herein may be attached to the appropriate portion of the subject's body and then used to measure the subject's ability to move the body part. Exemplary body parts for study include the upper extremity, specifically, the wrist, the shoulder, and the elbow, and the lower extremity, such as the ankle. Changes in the subject's ability to move may reflect the subject's response to therapeutic intervention before, during, or after a measurement, such as a response to drug therapy.

Exemplary attachment devices (wrist attachment, shoulder-elbow-wrist attachment, and ankle interface) are described above. A wide variety of variations in these devices are contemplated, and the reader is referred to the patent and patent applications incorporated herein. For example, systems may include one or more motors to provide at least one of assistance, perturbation, and resistance to an upper extremity motion. The wrist attachment, in particular, may include one or more motors to provide actuation about one or more of the pronation/supination, flexion/extension, and adduction/abduction axes of the wrist. For example, the wrist attachment may include a transmission system. The transmission system may include a pronation/supination (PS) motor coupled to a PS slide ring, such that the slide ring is rotatable about a PS axis and in a plane perpendicular to the forearm support long axis. The transmission system may also include a differential mechanism. In turn, the differential mechanism may include a first differential motor and a second differential motor. The differential mechanism may also include a gear system coupling the first and second differential motors to an arm which is rotatable with two degrees of freedom about a flexion/extension (FE) axis and an abduction/adduction (AA) axis substantially perpendicular to the FE axis.

A wide variety of sensors are contemplated here and are described in the incorporated patent applications. One or more sensors may be included in the transmission system. Sensors include motion sensors, torque sensors, and force sensors. One example of a sensor is an optical encoder. An optical encoder may be mounted on a motor shaft and provide a signal indicative of the rotational state of a motor.

Sensors produce signals indicative of some state of the subject and/or the attachment. For example, a signal may be indicative of a motion of the upper-extremity attachment, such as the attachment's speed, direction, or range of motion. The attachment's motion is an indicator of the subject's ability to move the attachment. That is, the attachment's speed and direction is at least in part a result of forces exerted by the subject and gives an indication of the subject's neurological and/or musculoskeletal ability. The range of motion through which a subject can move an attachment is also such an indication, as is the number of repetitions of a motion that a subject can perform. A sensor may also produce a signal that is indicative of a force applied to the upper extremity attachment.

Additional measurements include changes in kinematic and kinetic variables during unconstrained point-to-point movements with the attachment, such as movement displacement, movement duration, deviation from a straight line, aim, mean speed, peak speed, mean-to-peak speed ratio (smoothness), jerk (smoothness), correlation between speed and minimum-jerk speed, square of the difference between speed and minimum-jerk speed, number and magnitude and duration and overlap of submovements, and forces/power flow during constrained measurements.

An upper extremity attachment may also include a shoulder-elbow motion device, as shown in FIG. 2 and described above and in U.S. Pat. No. 5,466,213 and U.S. application Ser. No. 10/976,083. The shoulder-elbow motion device may include a member assembly having at least one degree of freedom and a distal free end to which the wrist attachment is coupled with at least one degree of freedom. U.S. application Ser. No. 10/976,083 describes a variety of ways in which the wrist attachment may be connected to a shoulder-elbow motion device. The shoulder-elbow motion device may include a drive system coupled to the member assembly. The drive system may include a shoulder motor coupled to one of the joints and controlling motion of the shoulder joint. The drive system may further include an elbow motor coupled to one of the joints and controlling motion of the elbow actuation joint.

Ankle-based attachments, described above and in U.S. Provisional Application No. 60/613,421, may also be used to measure changes in neurological and/or musculoskeletal performance. In some embodiments, both an upper-extremity attachment and an ankle interface can be used, sequentially or simultaneously.

5. Methods

The systems disclosed herein may be applied to a wide variety of methods, including measuring a change in neurological and/or musculoskeletal performance, and assessing efficacy of a therapy, such as a drug therapy.

A method of measuring a change in neurological or muscular performance of a subject may include attaching an attachment to a subject (FIG. 6). The subject may then be directed to perform a motion. A first force imposed by the subject on the attachment or a first motion of the attachment is sensed. The subject's first response may then be compared to a second response to determine a change in the subject's neurological or muscular performance. If the attachment includes a motor, then the motor can be actuated to provide at least one of assistance, perturbation, and resistance to the subject's motion.

This method may be adapted to measuring the efficacy of a therapy provided to the subject, such as drug therapy and/or physical therapy. If drug efficacy is being studied, then typically at least one measurement is made before administering a drug and at least one measurement is made after administering a drug. The measurements may then be compared to determine whether the subject's neurological and/or musculoskeletal performance has changed. When suitably controlled (i.e., by also performing the measurements on a subject who has not received the therapy), this method allows one to measure the efficacy of the drug of interest. The method may be particularly well applied to studying the efficacy of drugs used to help subjects improve neurological and/or musculoskeletal function, especially following an injury or during a degenerative disease. Exemplary conditions include stroke (both acute and chronic), spinal cord injury, traumatic injury to brain and/or spinal cord, Alzheimer's Disease, Huntington Disease, Parkinson's Disease, multiple sclerosis, amyotrophic lateral sclerosis, and many other neurological, neuromuscular, musculoskeletal, and other disorders, such as collagen vascular disease (rheumatoid arthiritis, systemic lupus erythematosus, scleroderma, dermatomyositis, and polyarteritis nodosa), arthritis, balance and gait disorders, and pain. Drugs are discussed in more detail below.

A method of conducting an efficacy study (FIG. 7) may include selecting a first and second group of subjects. A pre-administration property of each subject may be measured by attaching an attachment device (such as an upper-extremity attachment and/or an ankle interface) to the appropriate body part of the subject and sensing a property indicative of each subject's neurological or musculoskeletal function. A first therapy (which may be any type of therapy disclosed herein, including drug, biological, physical, or combinations of the above) may be administered to the subjects in the first group. A second therapy, placebo therapy (such as a therapy not believed to have any therapeutic effect), or no therapy may be administered to the subjects of the second group. A post-administration property of each subject may be measured by attaching the attachment mentioned above to each subject and sensing a property indicative of each subject's neurological or musculoskeletal function. A comparison may be made between pre-administration properties to post-administration properties measured from subjects in the first group to determine an efficacy of the first therapy. A similar comparison may be made in the second group to determine an efficacy of the second therapy, placebo, or no therapy. A comparison of the efficacy of the first therapy to the efficacy of the second therapy, placebo, or no therapy may also be made. This last comparison may be used to determine whether the first therapy is superior to the second therapy, placebo, or no therapy. In some cases, the second group may receive a second therapy to allow comparison of the efficacy between the first therapy and the second therapy. If the therapies are drug or biological therapies, this may be useful for purposes of determining the bioequivalency of the first drug (such as a generic drug) to a second drug (such as a marketed drug) in generating data that is required by the FDA when reviewing the first drug.

Efficacy trials may be controlled (i.e., one group receives the investigational therapy, and another group receives no therapy, a placebo, or a state-of-the-art therapy). They may be randomized to help normalize the test groups as to factors such as gender, age, ethnic background, behavior, and risk factors. Trials may also be blinded to prevent subjects from knowing whether they are receiving the therapy of interest (single blinded) and to prevent caregivers from knowing this as well (double blinded). If study groups cannot be made large enough to eliminate statistical disparities, then participants can be matched on one or more criteria (such as age and gender). Wherein the trial may include two groups, the first group may receive the therapy and the second group may receive the placebo or nothing. This allows the difference in the experimental group to the second group to be determined in order to determine therapy efficacy.

The system mentioned above can also be used to compare the data between two or more individuals. For example, the system can be used to determine whether a therapy's beneficial effect is greater for certain subjects than for others.

6. Therapeutic Treatments

A wide variety of therapeutic treatments are used to treat neurological and musculoskeletal disorders. Broad categories of treatments include drugs, biologicals (peptides, proteins, nucleic acids, vaccines, viruses, cells, stem cells, neural stem cells, hematopoietic stem cells, progenitor cells, neural progenitor cells, hematopoietic progenitor cells, tissue), human-administered physical therapy, and device-administered physical therapy (such as with the attachments and motion devices disclosed herein). Treatments may be combined; for example, a drug may be combined with a another drug, or with a biological (such as stem cells), or with a physical therapy. Combinations may be simultaneous (given at the same time), sequential (given one after the other), or given at defined intervals. Combinations of drugs and/or biologicals may be admixed for administration together. Administration of drugs and/or biologicals can be by any route of administration, including per os and parenteral (topical, intravenous, intramuscular, subcutaneous, intra-arterial, intrathecal, intrapleural, intraperitoneal, intrarectal, intravesical, intralesional).

Drugs typically used to treat Alzheimer's disease or related symptoms include cholinesterase inhibitors (such as tacrine and donepezil), rivastigmine, galantamine, galanthamine, memantine, metrifonate, bryostain, methylxanthine, non-steroidal anti-inflammatory drugs (rofecoxib, naxopren, celecoxib, aspirin, ibuprofen), vitamin E, selegiline, estrogen, ginkgo biloba extract, antidepressants, neuroleptics and mood stabilizers.

Drugs typically used to treat pain include analgesics (acetaminophen, acetaminophen with codeine, hydrocodone with acetaminophen, morphine sulfate, oxycodone, oxycodone with acetaminophen, propoxyphene hydrochloride, propoxyphene with acetaminophen, tramadol, tramadol with acetaminophen) and non-steroidal anti-inflammatory drugs (NSAIDs; diclofenac potassium, diclofenac sodium, diclofenac sodium with misoprostol, diflunisal, etodolac, fenoprofen calcium, flurbiprofen, ibuprofen, indomethacin, ketoprofen, meclofenamate sodium, mefenamic acid, meloxicam, nabumetone, naproxen, naproxen sodium, oxaprozin, piroxicam, sulindac, tolmetin sodium, choline and magnesium salicylates, choline salicylate, magnesium salicylate, salsalate, sodium salicylate).

Drugs typically used to treat ALS or related symptoms include riluzole, baclofen, tiranadine, dantrolene, benzodiazepines (such as diazepem), gabapentin, NSAIDs, cox2 inhibitors, tramadol, antidepressants, selective serotonin re-uptake inhibitors, selective dopamine blockers, branch-chain amino acids, phenytoin, quinine, lorazepam, morpine, arimoclomol, and chlorpromazine.

Drugs typically used to treat Parkinson's disease or related symptoms include levodopa, carbidopa, selegiline, bromocriptine, pergolide, amantadine, trihexphenidyl, benztropine, COMT inhibitors (catechol-O-methyl transferase), anticholinergics, dopamine precursors, dopamine receptor agonists, MAO-B inhibitors, and peripheral decarboxylase inhibitors.

Drugs typically used to treat Huntington's disease or related symptoms include neuroleptic agents, dopamine receptor blockers (such as haloperidol and perphenazine), presynaptic dopamine depletors (such as reserpine), clozapine, antidepressants, mood stabilizer, and antipsychotic agents.

Drugs typically used to treat multiple sclerosis or related symptoms include interferon beta-1 a, interferon beta-1b, glatiramer, mitoxantrone, natalizumab, corticosteroids (such as prednisone, methylprednisolone, prednisolone, dexamethasone, adreno-corticotrophic hormone (ATCH), and corticotropin), chemotherapeutic agents (such as azathiprine, cyclophosphamide, cyclosporin, methotrexate, cladribine), amantadine, baclofen, meclizine, carbamazepine, gabapentin, topiramate, zonisamide, phenyloin, desipramine, amitriptyline, imipramine, doxepin, protriptyline, pentoxifylline, ibprofen, aspirin, acetaminophen, hydroxyzine, antidepressants, and antibodies that bind to α-4-integrin (b1 and b7), e.g., TYSABRI® (natalizumab).

Compounds typically used to treat chronic stroke include benzodiazepines (such as midazolam), amphetamines (such as dextroamphetamine), type IV phosphodiesterase inhibitors (such as rolipram), type V phosphodiesterase inhibitors (such as sildenafil), and HMG-coenzyme A reductase inhibitors (such as atorvastatin and simvastatin) and nitric oxide donors, especially indirect nitric oxide donors. Other drugs of interest in treating stroke include inhibitors of mitochondrial permeability transition such as heterocyclics (methiothepin, mefloquine, propiomazine, quinacrine, ethopropazine, cyclobenzaprine, propantheline), antipsychotics (trifluoperazine, triflupromazine, chlorprothixene, promazine, thioridazine, chlorpromazine, prochlorperazine, perphenazine, periciazine, clozapine, thiothixene, pirenzepine), antidepressants (clomipramine, nortriptyline, desipramine, amitriptyline, amoxepine, maprotiline, mianserin, imipramine, doxepin), and antihistamines (promethazine, flufenazine, pimethixine, loratadine), mitochondial uncouplers such as 2,4-dinitrophenol, and antineoplastic drugs such as DNA intercalators (mithramycin).

Drugs typically used to treat acute stroke and spinal cord injury include thrombolytics (tissue plasminogen activator, alteplase, tenecteplase, and urokinase), antiplatelet agents (aspirin, clopidogrel, abciximab, anagrelide, dipyridamole, eptifibatide, ticlodipine, tirofiban), and anticoagulants (warfarin, heparin).

Drugs typically used to treat arthritis include cox2 inhibitors (etoricoxib, valdecoxib, celecoxib, rofecoxib), NSAIDs, and analgesics.

Drugs typically used to treat rheumatoid arthritis include auranofin, azathioprine, chlorambucil, cyclophosphamide, cyclosporine, gold sodium thiomalate, hydroxychloroquine sulfate, leflunomide, methotrexate, minocycline, penicillamine, sulfasalazine, TNF inhibitors (adalimumab, etanercept, infliximab), IL-1 inhibitors

(anakinra), and corticosteroids (betamethasone, cortisone acetate, dexamethasone, hydrocortisone, methylprednisolone, prednisolone, prednisolone sodium phosphate, prednisone).

Drugs typically used to treat fibromyalgia include NSAIDs, analgesics, and antidepressants (amitriptyline hydrochloride, duloxetine, fluoxetine).

The drugs described above can be combined with one another and with other substances. Combination therapies include conjoint administration with nicotinamide, NAD⁺ or salts thereof, other Vitamin B3 analogs, and nicotinamide riboside or analogs thereof. Carnitines, such as L-carnitine, may be co-administered, particularly for treating cerebral stroke, loss of memory, pre-senile dementia, Alzheimer's disease or preventing or treating disorders elicited by the use of neurotoxic drugs. Cyclooxygenase inhibitors, e.g., a COX-2 inhibitor, may also be co-administered for treating certain conditions described herein, such as an inflammatory condition or a neurologic disease.

A combination drug regimen may also include other agents or compounds for the treatment or prevention of neurodegenerative disorders, including Alzheimer's disease, ALS, Parkinson's disease, Huntington's disease, multiple sclerosis or secondary conditions associated with any of these conditions.

### EXAMPLES

The present description is further illustrated by the following examples, which should not be construed as limiting in any way. The contents of all cited references (including literature references, issued patents, published patent applications as cited throughout this application) are hereby expressly incorporated by reference. These examples are directed to testing the efficacy of sildenafil using a shoulder-elbow motion device but could be readily adapted to testing other drugs and other therapies using other attachments.

### Example 1

### Measuring Efficacy of Sildenafil in Stroke Recovery

The methods described herein can illuminate the relationship between recovery after stroke and normal motor learning. We have demonstrated the effectiveness of task specific training with robotic devices disclosed herein on motor recovery of the upper limb in patients with stroke. In controlled randomized trials, patients treated with robotic devices have improved motor outcome. We have also demonstrated how to use robotic devices disclosed herein to analyze motor performance and measure motor learning.

In patients with chronic stroke, it is a goal to test whether target drugs enhance motor learning, whether pharmacological treatment enhances motor recovery after stroke and, in a task specific training trial test whether pharmacological treatment interacts positively with robotic training. This phased set of experiments will rely on a robotic attachment as a kinematic measuring device and as a training device. Example 1 concerns the first of these phases.

Recent animal experiments indicate several classes of pharmacological agents that may influence the motor outcome after ischemic injury, without preference among the candidate mechanisms. Specifically in an embolic middle cerebral artery animal model of stroke, sildenafil improved unconventional measures of a transient motor deficit (Zhang, R., Y. Wang, L. Zhang, Z. Zhang, W. Tsang, M. Lu, et al., Sildenafil (Viagra) induces neurogenesis and promotes functional recovery after stroke in rats. Stroke, 2002. 33(11): p. 2675-80). That sildenafil improved motor outcome after stroke has captured the imagination of clinicians despite provocative shortcomings in the animal study. A further goal is to investigate first the effect of sildenafil on the kinematics of motor learning in patients with chronic stroke and in controls (non-neurologically injured age- and gender-matched).

A. Test Whether Sildenafil Improves Motor Learning.

These experiments will occur in patients with chronic stroke who are at least 6 months after stroke and in age- and gender-matched persons with no neurological injury. Patients and controls will be randomized to drug treatment or placebo. We will establish learning conditions in which a reproducibly applied force field generated by the robot will perturb the limb. The robotic device will measure the kinematics of the subsequent movement adaptation. Extensive studies in unimpaired subjects have shown that exposure to novel force fields generated by a robotic device evokes an adaptation that over the course of a few hundred movements systematically restores the pre-perturbation kinematic pattern and that kinematic variables provide an exquisitely sensitive measure of the course of motor learning. Consequently we will measure deviation of speed and path from the optimum unperturbed trajectory before, during and after the force field is applied. Experiments in control subjects will test whether sildenafil influences normal motor learning of a dynamic task. They will also provide a baseline against which to measure the performance of patients with chronic stroke. Data analysis will quantify the motor learning performance of patients with chronic stroke; test whether it differs from neurologically normal subjects and whether it is influenced by the drug treatment. Studies can also be directed to assessment of motor learning retention or motor forgetfulness. The outcome of this experiment will contribute to an understanding of the relation between learning and recovery after stroke and provide an initial assessment of the effect of drug treatment on both. Because the learning experiment can be completed quickly, it facilitates a progressive approach. This design can be repeated to test different doses or to test other candidate drugs, other therapies (such as physical therapy, robotic therapy, and various combinations of drugs, therapies, and drugs with therapies).

B. Motor Learning Rationale and Preliminary Data.

The sensitivity of the robotic method is such that the course of motor learning can reliably be measured in individual subjects. Groups of 8 or 9 subjects have proven sufficient to discriminate significant differences. Using the robotic device, we have found that in normals there is a correlation between the regional cerebral blood flow and progression through the motor learning tasks. Data suggest that early motor learning, during the initial force field perturbation, was associated with increased regional cerebral blood flow in the ventral striatum and the ipsilateral cortical brain areas. Late motor learning was associated with a shift of regional blood flow to contralateral hemisphere motor cortex and cerebellum. Also it is important to note that using these techniques we have found significant differences in patients with Parkinson's disease compared to normals.

It is expected that sildenafil will influence aspects of motor learning in normals. Comparisons of the results in normals to the effect sildenafil has on motor learning in patients with stroke will be determined. Possible outcomes include positive effects on learning that are enhanced in patients with stroke; a specific effect in patients with stroke and not in normals; or vice versa. If sildenafil has an effect in normals but no effect in patients with stroke the details of stroke patients' failure will be instructive. If sildenafil has no effect on motor learning in either group, then more effective compounds could be run through the experimental protocol. Alternatively, dose may be a crucial variable. An advantage of the phased approach is that, beginning with a motor learning study in Example 1, rapid quantification of the most promising candidate drug and dose before proceeding to a treatment trial according to Example 2 can be determined.

C. Motor Learning Protocol.

Patients will have chronic stroke and will come from a list of over 50 outpatients currently part of the waiting lists for robotic treatment at the Burke Rehabilitation Center. A patient will be included in the study if they meet the following criteria:
first single focal unilateral lesion with diagnosis verified by brain imaging (MRI or CT scans) that has occurred at least 6 months prior;
cognitive and language function sufficient to understand the experiments and follow instructions;
Motor Power score≧⅕ or ≦⅘ (neither complete paralysis nor fully recovered motor function in the 14 muscles of the shoulder and elbow); and
informed written consent to participate in the study.

Patients will be excluded from the study if they have:
a fixed contraction deformity in the affected limb;
a complete and total flaccid paralysis of all shoulder and elbow motor performance; or
an unstable (>5% change) baseline assessment across the three motor impairment scales (F-M, MSS, MP).

It is reasoned that nearly normal motor power would indicate nearly complete recovery and confound our study due to ceiling effects. Conversely, we expect that complete flaccid paralysis that persisted 6 months after stroke would be impervious to training, hence complete flaccid paralysis is an exclusion criterion. Along similar lines it would also be implausible to expect that any sensory motor training would have an impact on a fixed contraction deformity.

Patients will undergo three clinical evaluations at 2-week intervals in a one-month period. Patients will be excluded from the study if the clinical measures are not stable (<5% change) as this would indicate that they are not truly in the chronic phase of recovery after stroke.

Experience with standard, reliable and valid measures of neurological deficit include the sub-section of the Fugl-Meyer scale for shoulder/elbow and coordination (FM-SEC, 42 out of 66), Fugl-Meyer scale for wrist/hand (FM-WH; 24 out of 66), Motor Power (MP, maximum score=20), Motor Status score for shoulder and elbow (MS-SE, maximum score=40), and Motor Status score for wrist and hand (MS-WH, maximum score=42). The FM-SEC, -WH and the MS-SE, -WH measure quality of movement and subcomponents of functional movement. The MP measures strength in proximal muscles of the arm. A patient's score comprised the sum of the sub-scores.

The basic setup requires that a subject, seated in front of a robotic device, grasps a handle on the end of the robotic arm and moves it in the horizontal plane. A video screen is suspended above the robotic device and a cursor directs the movement from a central home point to distal points which match points on the planar surface in front of the subject. For the motor learning experiment trials may include a subject moving a screen cursor controlled by the robot end-effector between a centrally placed “home” target and 4 outward targets, which are pseudo-randomly presented. Trials are grouped into blocks of 80 movements (out and back 10 times from home to each of 4 targets) with varying environmental conditions as follows. In the first block the robot generates no forces (a “null” field condition) but records movement trajectories. This serves to establish a baseline unperturbed performance for each subject. (Other arrangements of video display and motion targets may be used with other attachments.)

In the second through fifth blocks of movements the robot generates a force field that perturbs the limb from its baseline trajectory. Specifically, the robot generates force proportional to the speed of hand movement but directed at right angles to it. As a result, the robot does no mechanical work on the subject, neither accelerating nor retarding the motion. The effect of this force field is to displace the hand trajectory laterally from its unperturbed path but, because the force vanishes when the limb stops moving, it does not prevent the subject from acquiring the target. The magnitude of the lateral displacement is determined by the proportionality constant relating force to velocity (the “strength” of the field). Field strength is adjusted (“titrated”) for each subject on the basis of behavior observed in the null field (first block of trials) to ensure that comparable initial perturbations are evoked from all subjects. From past experience, exposure to the force field over four blocks of trials (320 moves) is sufficient to evoke nearly complete adaptation so that the hand trajectories are restored to their pre-perturbation kinematics. Kinematic measures such as mean-squared lateral deviation of the hand path from a straight line connecting the start and target positions, and others described above, will allow us to quantify both the extent of adaptation and the rate of motor learning. If subjects show adaptation that is incomplete after 320 moves, additional blocks of trials in the force field may be added. This can be done seamlessly without excessively prolonging the experimental protocol.

In a final block of trials the force field is again removed (without informing the subjects). This serves to assess the after-effect of learning to compensate for the perturbation (normally evident as a movement displaced laterally in the direction opposite to that initially induced by the force field) and the rate of re-learning the unperturbed trajectory (which is typically complete within a single block of trials in the null field).

Subjects may be retested at a later time (for example, at least 24 hours later) to assess motor memory consolidation or motor memory retention.

There is extensive theoretical and experimental precedent for the effectiveness of this procedure to evoke motor learning. In the disclosed example, the entire sequence requires 6 blocks of trials (1 null field block, 4 force field blocks, 1 null field block) or 480 moves. Other sequences may include different numbers of blocks as well as negative transfer blocks. Negative transfer blocks may include force fields different from or directed in opposite directions from those in the force field blocks. They may be used to test whether exposure to the negative transfer block forces affect a subject's ability to learn to move in the presence of the regular force field block forces.

To avoid fatigue and boredom, subjects are allowed to rest briefly between blocks. The entire procedure typically occupies less than an hour for each subject. As a conventional therapy session typically lasts no longer than an hour, this procedure is well suited to the evaluation of stroke patients.

D. Motor Learning Data Analysis.

This design generates several learning conditions: unperturbed movement (null field), early phase field learning (first block with non-null field), late phase field learning (last block with non-null field) and after effect of field learning (last block). Throughout all trials we measure the complete kinematic response (i.e., hand path and time-course of motion) from which we derive multiple kinematic measures of learning and performance. There is both a theoretical and experimental basis to expect that subjects will learn to compensate for the force field to execute a maximally-smooth trajectory to the target (with straight hand path and symmetric, unimodal speed profile). Therefore, the principal measures of learning we will compute are the mean-squared lateral deviation of the hand path from the maximally smooth straight path between points; and the mean-squared deviation of the speed profile from the maximally-smooth (minimum-jerk) speed profile. We will also compute other measures that may provide detailed insight about possible origins of deviation from unperturbed performance. These include mean-squared angular deviation from the maximally smooth path; peak speed; maximum movement range, and the existence and progression of submovements. By adjusting the field strength as described above to evoke comparable initial deviations from the unperturbed path this design will provide a graded movement challenge for patients with chronic stroke and for normal subjects. Further, this will provide a comprehensive profile of kinematic performance that should be sensitive to identifying an effect of drug treatment.

E. Motor Learning. Power Analysis.

The lack of information about motor learning in patients with stroke on robotic generated tasks makes estimates of power speculative. We have had experience with the learning protocol as delivered by the robot. In brief, 8 patients who had Parkinson's disease demonstrated significant impairment in learning the trajectory of the unperturbed path (after the force field had been removed). It is noteworthy that the mean peak speed for the Parkinson's patients was comparable to the age matched control. This kinematic measure of deviation from the maximally smooth straight path between points was 4 fold depressed in the Parkinson's patients compared to 9 non-patients who were age matched (P<0.03). In 8 healthy volunteers we also showed significant motor learning during the blocks in which the force field perturbs the point-to-point movement and during the final block when the force field is removed and the subject must re-learn the unperturbed trajectory. We believe that the mean-squared lateral deviation of the hand path from the maximally smooth straight path between points will be sensitive to detect the influence of pharmacological agents on motor learning. Alternatively, we also have additional kinematic measures that may also be sensitive to detect a drug effect. Whether one or another measure is more sensitive is an empirical question.

### Example 2

### Efficacy of Sildenafil or Task Specific Robotic Treatment or Both in Promoting Motor Recovery in Patients with Chronic Stroke

It may be that molecular drug effects on recovery are not detected until they are combined with and guided by task specific behavioral manipulations and interaction with the environment. Alternatively, drug effects alone might alter the biological substrate permanently so that a single treatment schedule without environmental control may be sufficient to enhance recovery. The proposed design will maintain the treatment groups, so that a patient treated with drug in the learning experiment according to Example 1 remains on the drug in the recovery experiment according to Example 2. Information from Example 1 will inform the drug choice and dose for the recovery experiment.

Patients with chronic stroke who were treated with sildenafil for the first study will be randomly assigned to task specific robot treatment or conventional physical therapy. Those patients who were treated with placebo will also be randomly assigned to robot or conventional treatment. Patients will receive sildenafil or placebo daily, and, on task specific treatment days, one hour prior to robotic or conventional treatment. Patients will receive task specific robotic or conventional therapy 3 days a week for 6 weeks. There will be three clinical evaluations at 2-week intervals in the month prior to the start of robot or conventional treatment to ensure a stable deficit for the start point of the study. There will be a mid-point, end study and 6 month follow up clinical evaluation. At the end of treatment, a robotic test of motor learning performance will be administered (using the protocol described in Example 1). Data analysis will compare the clinical motor measures across the four treatment groups: sildenafil plus robotic therapy, sildenafil plus conventional therapy, placebo plus robotic therapy, placebo plus conventional therapy.

A. Motor Recovery Rationale and Preliminary Data.

We have recently demonstrated in patients with chronic stroke that task specific robotic training of the shoulder and elbow significantly changes the motor outcome (Ferraro, M., J. J. Palazzolo, J. Krol, H. I. Krebs, N. Hogan, and B. T. Volpe, Robot-aided sensorimotor arm training improves outcome in patients with chronic stroke. Neurology, 2003. 61 (11): p. 1604-7; Fasoli, S. E., H. I. Krebs, J. Stein, W. R. Frontera, and N. Hogan, Effects of robotic therapy on motor impairment and recovery in chronic stroke. Arch Phys Med Rehabil, 2003. 84(4): p. 477-82; MacClellan, L. R., D. D. Bradham, J. Whitall, B. T. Volpe, P. D. Wilson, J. Ohlhoff, et al., Robotic upper extremity neuro-rehabilitation for chronic stroke patients. Journal of Rehabilitation Research and Development, submitted). These studies included 107 patients. These patients were always at least 6 months post stroke and typically 3.5 years after stroke. While most change occurs after stroke within 12 weeks, these studies reflect the general outcome after conventional or alternate rehabilitation therapy and not task specific therapy. In addition to our studies using task specific robotic techniques, there is a growing literature that supports the notion that there are wider windows of opportunity for task specific training.

Treatment during the acute phase of stroke, whether the acute phase is considered in the acute hospital setting or in a rehabilitation setting within a month of stroke, may also be the subject of the present systems and methods. Efficacy of treatment during different windows before, during, and acutely after stroke may be assessed as described herein. For example, efficacy of hyperacute intervention within 3 hours or 6 hours of first symptoms can be studied by testing patients' ability to learn motor skills using extremity and joint attachments described herein. Efficacy of treatments during longer windows, such as 1 day to 1 week, 1 week to 4 weeks, and 4 weeks to 6 months post-stroke, may similarly be studied.

B. Motor Recovery Protocol.

Patients who have been randomized to drug treatment or placebo will retain their group status, and undergo further randomization to treatment with task specific robot training or conventional therapy. The fact that patients have a stable deficit supports a design without a no-treatment group. The stability of their pre-treatment deficit argues that with no treatment they would not change, and under these circumstances it would also be unethical to have a no-treatment group. Patients will take the sildenafil or placebo daily during the trial, and on the day of robotic or conventional treatment will take the medicine one hour before training.

The basic set up that defines the patient-robot relationship is identical to that in Example 1. The number and location of movement targets are as before but now, instead of a performance experiment in which the robot perturbs a movement, the robot will act to guide and correct movement or, in the case where a patient cannot make the movement, the robot will move the patient's limb. Treatment sessions will take approximately 1 hour and occur three times a week, for 6 weeks.

The control group will be treated with a specific protocol that exposes the patient to the identical number of treatments and time in extra treatment that robot treated patients receive. This protocol relies on general principles of therapeutic exercise applied to patients recovering from stroke. Briefly, the patient will experience a three stage program that includes: static stretching, active, assisted, and passive arm exercise and goal-directed planar reaching based on NDT/Bobath arm training.

The clinical assessment will include age, gender, side of stroke, the timing of stroke and the anatomical characteristics of the stroke. There are a variety of secondary measures that include disability as well as associated medical co-morbid conditions (for example, hypertension, coronary artery disease, diabetes mellitus, infection and depression) as well as the presence or development of pain in the shoulder and elbow. We routinely record and store this information. It will not be part of the analysis for the primary outcome impairment change data, but will be available for future studies.

A therapist blinded to the patients' group assignment and who was not the treating therapist will measure the neurological deficit in terms of impairment scores used in Example 1 to define a stable neurological deficit. These clinical measures will be obtained at the start of training, repeated at mid-point, at the end of the training and again in a 6 month follow up evaluation.

There will be an additional measure of motor learning taken at the end of the training. This protocol will be identical to Example 1.

C. Motor Recovery Data Analysis.

The statistical analysis will be an intent-to-treat analysis. Descriptive statistics will be compiled for each time point. The time points to be compared will be baseline, mid-session, end of treatment and 6 months after stroke. The paired t-test (and nonparametric Wilcoxon signed-rank test) will be used to evaluate mean score changes from baseline in the outcome scales for the four treatment groups [sildenafil plus robot; sildenafil plus conventional therapy; placebo plus robot; placebo plus conventional therapy; i.e., within-group comparisons over time]. The independent two-sample t-test (and nonparametric Wilcoxon rank-sum test) will be used to evaluate mean score differences among the groups (immediately after treatment and after 6 months follow-up; i.e., between group comparisons at each post-baseline time point). Similar analyses will be repeated for the secondary measures. The randomization of patients in the past has led to groups of comparable initial (baseline) impairment scores and we expect the intervention and control groups to be comparable after randomization on all other factors.

A repeated measures analysis of variance (RMANOVA) with group as a between-subject factor and time (baseline, after completion of treatment, and 6-month post stroke follow up) as a within-subject factor will be performed. Because we have repeated measures over time (i.e., a stable baseline and three time points of interest), this analysis will allow us to assess potential treatment-by-time interactions. In future studies additional variables of interest (i.e., stroke severity/location, time since stroke, depression, among other important secondary outcome assessments) can be modeled as covariates in the RMANOVA thus allowing for a multi-variate analysis. Because we are aware that the impairment scales cannot be considered dimensional, meeting the requirement of measurably equal intervals between any two adjacent ranked values, we will also explore the use of generalized linear mixed (hierarchical) models.

We expect to confirm the finding that robot training improves the motor outcome when compared to those treated with the conventional therapy. The possibility of finding improvement in the sildenafil group will inform the issue of whether robot training can be enhanced, and whether sildenafil should be a part of post-stroke therapy in general.

There will also be additional information that correlates the results of the motor learning study with the results of the motor recovery study. Patients who demonstrate greater learning capacity as indicated by kinematic performance may be more likely to experience further motor recovery and this improvement may be enhanced with sildenafil. Alternate outcomes derived from contrasting the results from the learning experiment with the recovery experiment should inform the relationship between learning and recovery. Initial data analysis will be binary; either learning occurred, or learning occurred in the presence of sildenafil. If learning occurred, did it predict recovery? This information might lead to the characterization of a surrogate marker of recovery derived from robotic measures of learning performance. If so, it would facilitate a phased approach to testing other drug treatments, allowing candidate drugs to be screened rapidly for their potential to enhance recovery before embarking on extensive treatment trials.

Alternatively, the lack of main effects or interactions will lead us to stratify the group on the basis of severity and lesion location. For example, the scores on the initial Fugl Meyer may be truncated to stratify the patients into a moderate and severe group. We have also had experience using the Canadian Neurological Scale and the Oxfordshire stroke location criteria to stratify patients, respectively, by initial stroke severity or lesion location. These classes of blocking strategy are reasonably straightforward with little potential for bias to affect group assignment.

D. Motor Recovery Power Analysis.

For the power analysis, data gathered in patients with chronic stroke is shown in Table 1 is used.

TABLE 1
Outpatients with moderate or severe stroke who were at least
6 months from the acute injury and who had robotic treatment
after demonstrating a stable deficit for two months.

| | | F-M S/E | MP | MSS S/E |
|---|---|---|---|---|
| | Measurement | (Max = 42; | (Max = 70; | (Max = 40; |
| Severity | Time | mean ± sem) | mean ± sem) | mean ± sem) |
| Moderate | Before Treatment | 16.8 ± 1.4 | 36.7 ± 2.4 | 24.6 ± 1.6 |
| (N = 14: | After Treatment | 22.2 ± 1.2 | 45.0 ± 1.5 | 27.3 ± 1.3 |
| CNS > 4; | Follow up | 25.4 ± 1.9 | 48.3 ± 2.6 | 28.3 ± 2.1 |
| NIHSS < | (3 mos) | | | |
| 15) | | | | |
| Severe | Before Treatment | 8.0 ± 0.6 | 16.8 ± 1.9 | 10.8 ± 1.1 |
| (N = 19 | After Treatment | 10.8 ± 0.8 | 23.3 ± 1.9 | 13.8 ± 1.1 |
| CNS < 4 | Follow up | 11.9 ± 1.8 | 24.2 ± 2.5 | 14.9 ± 2.0 |
| NIHSS > | (3 mos) | | | |
| 15) | | | | |

Power calculations were generated in PASS2000 (NCSS Statistical Software, Kaysville, Utah). These patients with chronic stroke were robot trained after three independent measures (over two months) demonstrated stable baseline performance. Since the patients with severe stroke, as judged by the Canadian Stroke Scale and the NIH Stroke Scale, would be excluded, we focused on the group with moderate stroke. We used the change measured at follow-up for the two clinical scales that demonstrated robust changes on the order of at least 20% improvement from the average value at the start of the trial. We estimated that a control group exposed to conventional therapy would improve about 10% compared to their initial measures, based on our experience with over 50 patients who were controls for an in-patient study. We believe this is a conservative estimate as these in-patients were participating in the standard post-stroke programs within a month of their stroke; a time interval during which the greatest changes are usually recorded. Twenty patients per group (robot, control) are required to detect the observed mean difference in FM S/E from the preliminary data with 85% power, and forty patients per group would generate 98% power. Twenty patients per group (robot, control) are required to detect the preliminary observed mean difference in MP with 95% power. To estimate group size to detect differences between sildenafil and placebo treatment requires an estimate of the effect of the drug. Because we can detect difference between the robot and control where the differences are ˜10%, and in view of the findings in the animal study of a transient 10% difference between sildenafil and control, we conservatively estimated that sildenafil treatment that caused a 10% improvement over the placebo group whether they had robot or conventional therapy should be the detection criteria. Forty patients per group (sildenafil, placebo) are required to detect the observed mean difference in FM S/E from the preliminary data with 90% power. Forty patients per group (sildenafil, placebo) are required to detect the preliminary observed mean difference in MP with 97% power. Instead of the MS S/E driving the overall sample size, we plan to rely on the FM S/E and the MP motor outcomes scores, since the small difference between the treatment protocols for MS S/E make the number of required patients too large to recruit in a timely fashion.

In general our plan is based on a progressive phased approach. First we will use the robot as an accurate measuring device to test whether learning can be influenced by pharmacological intervention. Then we will launch a treatment trial that is informed by the results of the learning trial in the initial experiment. We believe this stepwise approach will foster an understanding of the pharmacological intervention and its optimum dose and will maximize the chance of a favorable outcome of the recovery trial.

### Example 2

### Development of Sensitive n=1 Experiments in CNS Drug Development

The sensitivity of the measurements attainable with the disclosed attachments is such that the course of motor learning can reliably be quantified in individual subjects or in groups as desired. This approach allows for repeat measurements and longitudinal observations in individual subjects. The sensitivity of the robotic measurements will be useful as an indicator in a variety of Central Nervous System (CNS) pathologies.

It is therefore the objective to: (1) establish a supersensitive, reproducible protocol to quantify motor behavior of the arm, (2) to integrate this protocol with a scalable motor learning task so that motor performance will act as a biomarker for higher cognitive function, and (3) to apply both 1+2 to longitudinal observations of normal subjects and patients with CNS disease, including a period of pharmacological intervention.

A. Study Parts:

Planned studies may include the following steps: (1) Definition of a motor learning protocol−automate data analysis and graphical presentation; (2) Collection of normative data on (a) normal subjects, (b) patients with mild Alzheimer's Disease (AD), and (c) patients with a non-acute focal neurological deficit. The number of test subjects herein is 9, however this number can be any that is typically used in testing drug trials. There will be 3 observations at intervals of two weeks; and (3) Longitudinal observation over an additional 10 weeks in the same subjects/patients, with observations at intervals of two weeks. For the first 5 weeks patients will undergo a pharmacological treatment (AD: cholinesterase inhibitors, focal neurological deficit: indirect NO donors).

Our intention is to develop this biomarker to eventually enable N=1 experiments with repeat measures to build longitudinal models in “untreated” subjects, then introduce a treatment intervention, and finally withdraw the treatment intervention. The objective is to detect “disease modifying” treatment effects by showing that potential treatment benefits survive beyond withdrawal of therapy.

Patients and subjects entry criteria may include cognitive and language function sufficient to understand the experiments and follow instructions; motor power score≧⅕ or ≦⅘; and informed written consent to participate in the study.

B. Robotic Setup: Shoulder-Elbow Motion Device

For a setup involving a shoulder-elbow motion device, a subject, seated in front of a robotic device, grasps a handle on the end of the robotic arm and moves it in the horizontal plane. A video screen is suspended above the robotic device and a cursor directs the movement from a central home point to distant points which match points on the planar surface in front of the subject (total excursion 15 cm). A trial may include a subject moving a screen cursor matched by the robot end-effector between a centrally placed “home” target and 4 distant targets, which are pseudo-randomly presented. Trials are grouped into blocks of 80 movements (out and back 10 times from home to each of 4 targets) with varying environmental conditions as follows. In the first block the robot generates no forces (a “null” field condition) but records speed and movement trajectories. This serves to establish a baseline unperturbed performance for each subject.

In the second through fifth blocks of movements the robot generates a force field that perturbs the limb from its baseline trajectory. Specifically, the robot generates force proportional to the speed of hand movement but directed at right angles to it. As a result, the robot does no mechanical work on the subject, neither accelerating nor retarding the motion. The effect of this force field is to displace the hand trajectory laterally from its unperturbed path but, because the force vanishes when the limb stops moving, it does not prevent the subject from acquiring the target. The magnitude of the lateral displacement is determined by the proportionality constant relating force to velocity (the “strength” of the field). Field strength is adjusted (“titrated”) for each subject on the basis of behavior observed in the null field (first block of trials) to ensure that comparable initial perturbations are evoked from all subjects. Kinematic measures such as mean-squared lateral deviation of the hand path from a straight line connecting the start and target positions will allow us to quantify both the extent of adaptation and the rate of motor learning.

In a final block of trials, the force field is again removed (without informing the subjects). This serves to assess the after-effect of learning to compensate for the perturbation (normally evident as a movement displaced laterally in the direction opposite to that initially induced by the force field) and the rate of re-learning the unperturbed trajectory (which is typically complete within a single block of trials in the null field).

C. Follow-up Work

If successful, this biomarker could be deployed on other disease states such as schizophrenia, Multiple Sclerosis. The long-term goal is to develop a tool that can detect pharmacological modulation in a wide range of CNS disease states in individual subjects and allow longitudinal observations.

1. A system for measuring a change in neurological or muscular performance of a subject, comprising:
a joint and/or extremity attachment and/or motion device which includes a first support so sized and shaped as to be able to receive a portion of the subject's anatomy;
a sensor producing a first signal indicative of a force imposed by the subject on the attachment and/or device and/or of a motion of the attachment and/or device; and
a controller comprising a computation circuit, responsive to the first signal, to:
compare the first signal to a second signal, the second signal being produced at a different time from the first signal, the second signal being indicative of a force imposed by the subject on the attachment and/or device and/or of a motion of the subject and the attachment and/or device; and
generate an output signal indicative of a difference, if any, between the first signal and the second signal.

2. The system of claim 1, wherein the attachment and/or motion device further comprises a second support so sized and shaped as to be able to receive a portion of the subject's anatomy and a linkage connecting the two supports.

3. The system of claim 1, wherein the first signal is indicative of a motion of the attachment and/or device and/or of a force applied to the attachment and/or device.

4. A method of measuring a change in neurological and/or muscular performance of a subject, comprising:
attaching the attachment and/or motion device of the system of claim 1 to the subject;
directing the subject to perform a motion;
sensing a first force imposed by the subject on the attachment and/or device and/or a first motion of the attachment and/or device; and
comparing the first force and/or motion to a second force and/or motion to determine a change in neurological and/or muscular performance of a subject.

5. The method of claim 4, further comprising administering a therapeutic treatment to the subject.

6. The method of claim 5, wherein administering is performed between sensing the first force or motion and sensing the second force and/or motion.

7. The method of claim 5, wherein the therapeutic treatment comprises a neurological drug.

8. The method of claim 7, wherein the neurological drug comprises a stroke drug.

9. The method of claim 7, wherein the drug comprises a phosphodiesterase inhibitor.

10. The system of claim 1, wherein the attachment and/or device comprises a shoulder-elbow motion device comprising a member assembly having at least one degree of freedom and a distal free end comprising the first support.

11. The system of claim 1, wherein the attachment and/or motion device comprises an upper-extremity attachment including a wrist attachment, wherein:
the first support comprises a forearm support, so sized and shaped as to be able to receive a forearm of the subject, the forearm support defining a long axis; and
the attachment further comprises:
a second support including a handle so positioned in relation to the forearm support and so sized and shaped as to be able to receive the subject's hand;
a linkage connecting the two supports; and
a transmission system providing rotation with at least three degrees of freedom.

12. The system of claim 11, wherein the first signal is indicative of a motion of the upper-extremity attachment and/or of a force applied to the upper extremity attachment.

13. A method of measuring a change in neurological and/or muscular performance of a subject, comprising:
attaching the upper-extremity attachment of the system of claim 11 to the subject's upper extremity;
directing the subject to perform a motion;
sensing a first force imposed by the subject on the upper-extremity attachment and/or a first motion of the upper-extremity attachment; and
comparing the first force and/or motion to a second force and/or motion to determine a change in neurological and/or muscular performance of a subject.

14. The method of claim 13, wherein the upper extremity attachment further comprises a motor, and the method further comprises actuating the motor to provide at least one of assistance, perturbation, and resistance to an upper extremity motion.

15. The method of claim 13, further comprising administering a therapeutic treatment to the subject.

16. The method of claim 15, wherein administering is performed between sensing the first force or motion and sensing the second force and/or motion.

17. The method of claim 15, wherein the therapeutic treatment comprises a neurological drug.

18. The method of claim 17, wherein the neurological drug comprises a stroke drug.

19. The method of claim 17, wherein the drug comprises a phosphodiesterase inhibitor.

20. The method of claim 15, wherein the first force and/or motion is sensed before administration and the second force and/or motion is sensed after administration.

21. The system of claim 11, wherein the upper-extremity attachment further comprises a shoulder-elbow motion device comprising a member assembly having at least one degree of freedom and a distal free end to which the wrist attachment is coupled with at least one degree of freedom.

22. The system of claim 21, wherein the member assembly comprises:
an arm member coupled at its distal end to the proximal end of a forearm member by an elbow joint, the arm member and the forearm member rotatable with respect to one another about the elbow joint;
a third member coupled at its distal end to the midshaft of the forearm member by an elbow actuation joint, the third member and the forearm member rotatable with respect to one another about the elbow actuation joint; and
a fourth member coupled at its proximal end to the proximal end of the arm member by a shoulder joint, the fourth member and the arm member rotatable with respect to one another about the shoulder joint; the fourth member also coupled at its distal end to the proximal end of the third member by a fourth joint, the third member and the fourth member rotatable with respect to one another about the fourth joint.

23. The system of claim 22 further comprising:
a drive system coupled to a member assembly, wherein the drive system comprises:
a shoulder motor coupled to one of the joints and controlling motion of the shoulder joint; and
an elbow motor coupled to one of the joints and controlling motion of the elbow actuation joint.

24. The system of claim 21, wherein the first signal is indicative of a motion of the upper-extremity attachment and/or of a force applied to the upper extremity attachment.

25. A method of measuring a change in neurological and/or muscular performance of a subject, comprising:
attaching the upper-extremity attachment of the system of claim 21 to the subject's upper extremity;
directing the subject to perform a motion;
sensing a first force imposed by the subject on the upper-extremity attachment and/or a first motion of the subject and the upper-extremity attachment; and
comparing the first force and/or motion to a second force and/or motion to determine a change in neurological and/or muscular performance of a subject.

26. The method of claim 25, further comprising administering a therapeutic treatment to the subject.

27. The method of claim 26, wherein the first force and/or motion is sensed before administration and the second force and/or motion is sensed after administration.

28. The method of claim 25, wherein the therapeutic treatment comprises a drug.

29. The method of claim 28, wherein the drug comprises a neurological drug.

30. The method of claim 29, wherein the neurological drug comprises a stroke drug.

31. The method of claim 57, wherein the drug comprises a phosphodiesterase inhibitor.

32. The system of claim 1, wherein the attachment and/or device comprises an ankle interface, wherein:
the first support comprises a leg connection attachable to a user's leg; and
the ankle interface further comprises:
a second support including a foot connection attachable to the user's corresponding foot; and
a transmission system coupling the leg connection and the foot connection with at least two degrees of freedom.

33. The ankle interface of claim 32, wherein the transmission system comprises at least one motor providing actuation.

34. The system of claim 32 wherein the first signal is indicative of a motion of the ankle interface and/or of a force applied to the ankle interface.

35. A method of measuring a change in neurological and/or muscular performance of a subject, comprising:
attaching the ankle interface of the system of claim 32 to the subject's leg and foot;
directing the subject to perform a motion;
sensing a first force imposed by the subject on the ankle interface attachment and/or a first motion of the ankle-interface attachment; and
comparing the first force and/or motion to a second force and/or motion to determine a change in neurological or muscular performance of a subject.

36. A method of conducting a drug trial, comprising:
selecting first and second groups of subjects;
measuring a pre-administration property of each subject by attaching the joint and/or extremity attachment and/or motion device of the system of claim 1 to each subject, and sensing a property indicative of each subject's neurological and/or musculoskeletal function;
administering a first drug to the subjects in the first group;
administering a second drug, a placebo, or no drug to the subjects of the second group;
measuring a post-administration property of each subject by attaching the joint and/or extremity attachment and/or motion device of the system of claim 1 to each subject, and sensing a property indicative of each subject's neurological and/or musculoskeletal function;
comparing pre-administration properties to post-administration properties measured from subjects in the first group to determine an efficacy of the first drug;
comparing pre-administration properties to post-administration properties measured from subjects in the second group to determine an efficacy of the second drug, placebo, or no drug; and
comparing the efficacy of the first drug to the efficacy of the second drug, placebo, or no drug.
